(19) United States
(12) Patent Application Publication
(10) Pub. No.: US 2009/0208424 A1
(43) Pub. Date: Aug. 20, 2009

(54) COMPOSITIONS AND METHODS FOR DELIVERY OF SOLUTION TO THE SKIN

(76) Inventors: Todd Maibach, Menlo Park, CA (US); Brian C. Keller, Antioch, CA (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 12/033,618

(22) Filed: Feb. 19, 2008

(51) Int. Cl. A61K 9/12; A61P 29/00

(52) U.S. Cl. 424/45

(57) ABSTRACT

Compositions and methods for delivery of medicaments to human or animal skin, for example, the scalp, comprising the steps of transporting the medicaments in a volatile carrier solution to a position adjacent to the skin, and atomizing the preparation at said position onto the skin to thereby uniformly distribute the preparation over the scalp thereby improving absorption of the medicament through the skin

### FIELD OF THE INVENTION

The present invention relates to compositions and methods for delivering a solution to human or animal skin comprising the steps of transporting the medicaments such as an anti-inflammatory in a volatile carrier solution to a position adjacent to the skin, and atomizing the preparation at the position onto the skin to uniformly distribute and improve absorption of the medicament through the skin.

### BACKGROUND OF THE INVENTION

This invention relates to compositions and methods for delivering solution to the skin. Specifically, the invention also relates to compositions and methods for treating human or animal skin conditions and disorders by delivering medicinal solutions to the affected skin area in a novel manner. The invention may assist in various skin and scalp disorders such as dermatoses and seborrheic dermatitis.

Heretofore, various medicinal formulations have been made available for treating conditions such as dermatoses or specifically seborrheic dermatitis, which is associated with dandruff. The formulations have comprised both “anti-dandruff” shampoos and alcohol-based, glycol-based or similarly based solutions or ointments generally containing anti-inflammatory agents alone or anti-microbial agents such as hexachlorophene or benzalkonium chloride, or a combination of both anti-inflammatory agents and anti-microbial agents. Although these medicinal formulations have proved successful in treating various types of dandruff and dermatoses conditions, they have had the disadvantage of being inconvenient and/or difficult to apply.

One mode of applying “anti-dandruff” or “anti-dermatoses” formulations has been achieved by preparing them within a shampoo. This mode of application is unsatisfactory because while major amounts of the active components of the shampoos initially contact the scalp, rinsing out the shampoo eliminates substantial amounts of the active components of the shampoo and thereby prevents the shampoos from having significant or lasting medicinal effect.

Because of the substantial loss of active agents from the scalp during rinsing, it is economically impractical to provide shampoos with expensive components such as anti-inflammatory steroids, for example dexamethasone. The effect of this exclusion of expensive components is that the shampoos are significantly less effective than they would otherwise be if they contained these expensive components. Furthermore, substantial amounts of hair on the scalp may have a negative impact on shampoo formulations. Because of the total wetting of the hair by the shampoos, shampoos cannot be used as often as required, for example, two or three times a week for severe dandruff and/or seborrheic dermatitis conditions, by women who regularly have their hair styled.

The aforementioned alcohol- and glycol-based solutions may also be applied to the scalp using an eyedropper or by simply pouring the solution onto the scalp. These application methods localize, that is, non-uniformly distribute, the solution on the scalp so that there is a relatively high concentration of the solution and active components at the center of the applied solution with decreasing concentrations and effectiveness to the edge of the applied solution.

The solutions may also be applied using aerosol containers having a normal spray head. However, this method often results in only a small percentage of the solution finding its way to the scalp for treatment of the affected area. In cases of skin with substantial amounts of hair, matting of the hair may be caused by this method due to the large amounts of solution deposited on the hair. This method is very restricted in its use since it cannot be used on skin areas where the hair is relatively thick, such as exists on most women's scalps.

The aforementioned ointments have somewhat the same disadvantages as those associated with use of the above-mentioned solutions. Moreover, application with an ointment is inefficient and often very messy. The ointment is often applied with a finger, and only partial amounts of the ointment from the finger may be transferred to the intended area. Moreover, for skin with substantial amounts of hair, the hair must be parted to expose the affected area and, thereafter, the ointment is rubbed over the affected area. Besides the resulting localization of the ointment, substantial amounts of the ointment are coated onto the hair making the use of ointments inefficient for transferring the medicament to the intended area.

The patent to Winbray, U.S. Pat. No. 1,150,238 and the patent to Boghosian, U.S. Pat. No. 3,730,182 both describe a device somewhat similar in principle to that shown in the present invention. However, Winbray atomizes by passing air under pressure through a medicated liquid. Moreover, Boghosian relates to the use of an anti-inflammatory such as hydrocortisone and a propellant, dichlorotetrafluoroethane. Dichlorotetrafluoroethane, a chlorofluocarbon, (CFC) is a known to deplete the ozone. The present invention, however, utilizes an inert propellant such as heptafluoropropane, which does not include chorine, and is currently not known to deplete the ozone. Specifically, the present invention utilizes an inert non-CFC propellant such as heptafluoropropane in combination with a volatile alcohol such as isopropyl alcohol or ethanol to promote instant evaporation of the inert carrier at the skin, thus distributing an anti-inflammatory such as dexamethasone, or other medicament, onto the scalp in the form of micro-crystals which promotes rapid absorption and subsequent healing, as discussed more fully below.

### SUMMARY OF THE INVENTION

The present invention relates to compositions and methods for delivering a solution to human or animal skin in a volatile carrier solution to a position adjacent to the skin, and atomizing the preparation at the position onto the skin to distribute and improve absorption of the medicament through the skin.

In one embodiment, the present invention may include a composition comprising a pressurized aerosol spray container having an actuator and containing a composition comprising an effective amount of an anti-inflammatory agent, and a carrier comprising non-volatile means for solubilizing the anti-inflammatory agent, a volatile organic compound and heptafluoropropane, and having a nozzle having a bore extending therethrough and having an exhaust end and an opposite end communicating with the container.

In another embodiment, the present invention may comprise a composition wherein the volatile organic compound is ethanol.

In another embodiment, the present invention may comprise a composition wherein the anti-inflammatory agent is a steroid.

In another embodiment, the present invention may comprise a composition wherein the steroid is dexamethasone.

In another embodiment, the present invention may comprise a composition wherein the solubilizing means is an ester derived from a lower alkyl alcohol and a fatty acid. In another embodiment, the present invention may comprise a composition wherein the alcohol is isopropyl alcohol. In another embodiment, the present invention may comprise a composition wherein the fatty acid is myristic acid.

In one embodiment, the present invention may include a composition comprising a pressurized aerosol spray container having an actuator and containing a composition comprising isopropyl myristate, ethanol, heptafluoropropane, and an effective amount of dexamethasone, and having a nozzle having a bore extending therethrough and having an exhaust end and an opposite end communicating with said container.

In another embodiment, the present invention may comprise a composition wherein an effective amount of dexamethasone is about 0.01 percent by weight of the composition less the weight of heptafluoropropane.

In another embodiment, the present invention may comprise a composition wherein 0.02 mg of dexamethasone is dispensed per second.

In another embodiment, the present invention may comprise a composition wherein the amount of isopropyl myristate is about 31.99 percent by weight of the composition less the weight of heptafluoropropane.

In another embodiment, the present invention may comprise a composition wherein the nozzle has a length of about 5 inches and an outer diameter of about ⅛ inch.

In one embodiment, the present invention may include methods comprising the delivery of medicaments to human or animal skin areas comprising:
a pressurized aerosol spray container having an actuator and containing a composition comprising an effective amount of an anti-inflammatory agent, and a carrier comprising non-volatile means for solubilizing said anti-inflammatory agent, a volatile organic compound and heptafluoropropane, and having a nozzle having a bore extending therethrough and having an exhaust end and an opposite end communicating with the container; and placing the exhaust end of the nozzle adjacent the skin areas; and actuating the actuator such that the composition in the container is carried by the nozzle to the skin areas in atomized form, whereby the anti-inflammatory agent is concentrated in the non-volatile solubilizing means thereby facilitating absorption of the anti-inflammatory agent by the skin areas.

In another embodiment, the present invention may include methods comprising the compositions wherein the volatile organic compound is ethanol.

In another embodiment, the present invention may include methods comprising the compositions wherein the anti-inflammatory agent is a steroid.

In another embodiment, the present invention may include methods comprising the compositions wherein the steroid is dexamethasone.

In another embodiment, the present invention may include methods comprising the compositions wherein the solubilizing means is an ester derived from a lower alkyl alcohol and a fatty acid. In another embodiment, the present invention may include methods comprising the compositions wherein the alcohol is isopropyl alcohol. In another embodiment, the present invention may include methods comprising the compositions wherein the fatty acid is myristic acid.

In one embodiment, the present invention may include methods comprising the delivery of medicaments to human or animal skin areas comprising: a composition comprising a pressurized aerosol spray container having an actuator and containing a composition comprising isopropyl myristate, ethanol, heptafluoropropane and an effective amount of dexamethasone, and having a nozzle having a bore extending therethrough and having an exhaust end and an opposite end communicating with the container, and placing the exhaust end of the nozzle adjacent the skin area; and actuating the actuator such that the composition in the container is carried by the nozzle to the skin areas in atomized form, whereby the dexamethasone is concentrated in the isopropyl myristate thereby facilitating absorption of dexamethasone by the skin areas.

In another embodiment, the present invention may include methods comprising the compositions wherein an effective amount of dexamethasone is about 0.01 percent by weight of the composition less the weight of heptafluoropropane.

In another embodiment, the present invention may include methods comprising the compositions wherein 0.02 mg of dexamethasone is dispensed per second.

In another embodiment, the present invention may include methods comprising the compositions wherein the amount of isopropyl myristate is about 31.99 percent by weight of the composition less the weight of heptafluoropropane.

In another embodiment, the present invention may include methods comprising the compositions wherein the nozzle has a length of about 5 inches and an outer diameter of about ⅛ inch.

### BRIEF DESCRIPTION OF THE DRAWINGS

FIG. 1 is the view of one specific embodiment regarding an atomizer shown in operating position with respect to a person's scalp for carrying out the method of this invention.

FIG. 2 is a close-up of the nozzle of the atomizer of FIG. 1 with its exit end in operating position adjacent a person's scalp.

### DETAILED DESCRIPTION OF THE INVENTION

The compositions and methods of this invention comprise spraying or atomizing a medicinal or chemical solution or preparation directly onto affected skin areas. This is accomplished by directing the medicinal or chemical solution, at a controlled rate, onto the skin in atomized form from a point adjacent the skin. More specifically, the medicinal or chemical solution is atomized by and exhausted from an atomizer having a nozzle, the exhaust end of which is placed adjacent the skin when it is desired to uniformly cover the affected skin areas.

The term “adjacent” as used herein includes, nearby, nearly touching and touching of the skin with the end of the nozzle when angled thereto as shown in FIG. 2. The term “atomizer” as used herein includes, but is not limited to, the conversion of bulk liquid into a spray, mist, or a collection of drops. In a specific embodiment, the atomizer may be a pressurized spray container that assists in the conversion of bulk liquid into a spray, mist, or a collection of drops. In another specific embodiment, the pressurized aerosol spray container may have an actuator. In another specific embodiment, the atomizer may be used by by passing a liquid, such as a medicinal solution. In a specific embodiment, the medicinal solution may be transported to a position adjacent to the skin and directed onto the affected areas of skin in atomized form by an atomizer containing both the medicinal solution and a gaseous propellant. In a specific embodiment, the atomizer may be fitted with a nozzle. In another specific embodiment, the atomizer may pass the liquid or medicinal solution through a nozzle. In another specific embodiment, the atomizer is fitted with a small-diameter, elongated nozzle having a bore extending therethrough. Because of the narrowness and elongation of the nozzle, its working end can be placed close to the skin, which is to be treated, so that atomization of the solution takes place substantially at the skin surface.

In a specific embodiment, the compositions and methods of the invention may comprise spraying solution onto affected areas of the skin, regardless of the presence of hair on the skin. In a specific embodiment, the compositions and methods of the invention may comprise spraying solution onto affected areas of the scalp. In a specific embodiment, the compositions and methods of the invention may comprise spraying the solution onto affected skin areas when hair is present on the skin. Specifically, the compositions and methods of the invention may comprise spraying the solution onto affected skin areas having small to substantial amounts of hair. In another specific embodiment, the compositions and methods of this invention may be employed to treat hairy skin areas of both man and lower animals. In a specific embodiment, the compositions and methods of this invention comprise atomizing or spraying a medicinal preparation onto affected skin which may have small to substantial amounts of hair growing thereon without significantly wetting or coating the hair with the preparation. More specifically, a medicinal solution may be transported in a substantially closed system or confined zone past substantially all of the hair growing outwardly from the skin to a position adjacent to the skin where it is atomized and directed, in atomized form onto the skin. In this manner, the solution is transported past substantially all of the hair before being sprayed onto the underlying skin.

In a specific embodiment, the compositions and methods of the invention may be used to treat skin conditions. The advantages of treating skin conditions in this manner include the ability to uniformly direct substantially the desired amount of medicinal solution onto the affected skin areas. Additionally, only affected skin areas are treated; adjacent skin areas need not be contacted by the atomized medicinal solution.

Moreover, it is believed that penetration of the active ingredients in the medicinal composition into the skin or scalp may be greatly enhanced by transporting the medicinal composition to a position adjacent the scalp in a carrier comprising non-volatile means for solubilizing the medicament, e.g. isopropyl myristate and a volatile organic compound such as ethanol, isopropyl alcohol or aliphatic alcohols, and a non-CFC propellant, such as tetrafluoroethane and heptafluoropropane. Upon atomization, the volatile portions of the carrier evaporate leaving behind a supersaturated solution of medicament such as an anti-inflammatory agent in the non-volatile solubilizing means. The supersaturated solution of medicament acts as a driving force to promote rapid diffusion of the medicament to the skin. This result is due to the well-known principle of diffusion of substances from points of high concentration to points of low concentration. When the volatile portions of the carrier evaporate, the medicament such as an anti-inflammatory agent remains in the non-volatile solubilizing means in the form of microcrystals which must redissolve to be available for diffusion and absorption by the skin and thus have a longer healing effect.

In a specific embodiment, various skin disorders can be treated by the compositions and methods of the present invention, particularly the skin disorders dermatoses and seborrheic dermatitis. However, skin disorders that may be treated include, but are not limited to, dermatoses, dermatitis, seborrheic dermatitis, dandruff, and the management of abnormal raised skin scars. Specifically, the compositions and methods of the invention may be employed to treat corticosteroid-responsive dermatoses and/or the inflammatory skin conditions seborrheic dermatitis.

In a specific embodiment, the compositions and methods of the present invention may include an anti-inflammatory agent. In another specific embodiment, the anti-inflammatory agent may be dexamethasone. Dexamethasone is a corticosteroid that also has antipruritic effects. Therefore, dexamethasone may be useful for treatment and relief of skin conditions such as dermatoses and/or the inflammatory skin conditions seborrheic dermatitis when applied topically.

In another specific embodiment, the compositions and methods of the present invention may comprise a volatile organic compound. In a specific embodiment, the volatile organic compound may be ethanol.

In another specific embodiment, compositions and methods of the present invention may comprise a carrier comprising non-volatile means for solubilizing the anti-inflammatory agent. In a specific embodiment, the carrier with solubilizing means may be an ester derived from a lower alkyl alcohol and a fatty acid. In another specific embodiment, the lower alkyl alcohol may be isopropyl alcohol. In another specific embodiment, the fatty acid may be myristic acid. In another specific embodiment, the carrier with solubilizing means may be isopropyl myristate. In a specific embodiment, the compositions and methods of the present invention may be any solution lending itself to atomization or vaporization.

In another specific embodiment, the compositions and methods of the present invention may comprise an effective amount of an anti-inflammatory agent. The term “effective amount” means an amount of a compound/composition according to the present invention effective in producing the desired therapeutic effect.

In one specific embodiment, anti-inflammatory agent, a volatile organic compound and a carrier comprising non-volatile means for solubilizing the anti-inflammatory agent may be comprised in a solution. In one specific embodiment, the invention comprises atomizing a solution at a point adjacent the affected areas of the skin or scalp. That is, the solution is transported to a point adjacent the affected skin area within an enclosure before being released into the air in atomized form. The rate of atomization is controlled so that substantially only the desired amount of solution is directed onto the affected areas per unit of time.

In one specific embodiment, the solution is combined, under pressure, with a gaseous propellant in a container or atomizer having a nozzle or delivery tube. In another specific embodiment, the nozzle may be an elongated nozzle. The exhaust end of the elongated nozzle may be placed substantially in contact with the affected scalp area and the solution may be directed onto the affected areas while moving the exhaust end of the nozzle across the affected areas.

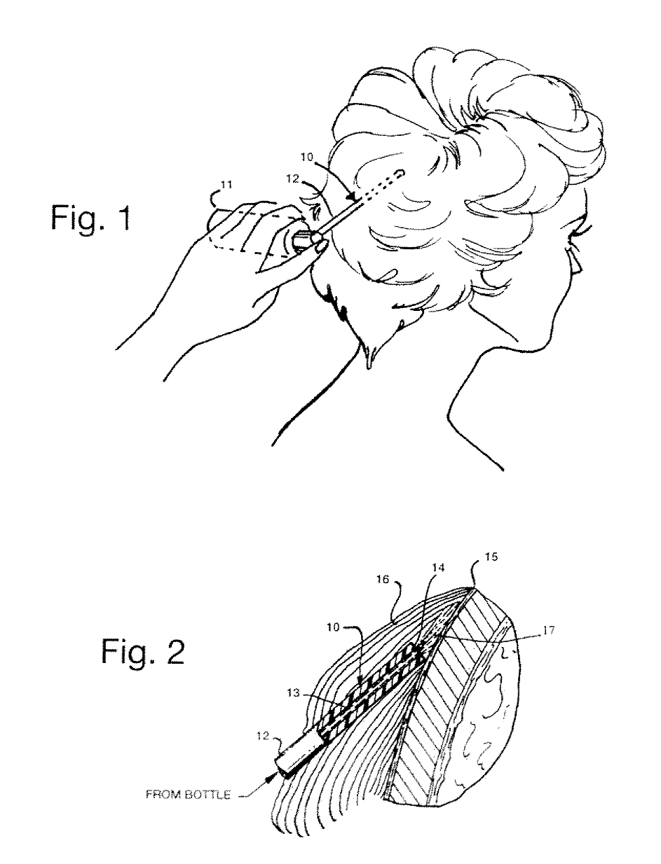

Referring now to the figures, FIG. 1 and FIG. 2 present one specific embodiment of applying the present invention to a scalp with hair, and in no way limits other embodiments of the present invention. Specifically in FIG. 1, numeral 10 designates a pressurized spray container or atomizer comprising a reservoir 11 and an elongated nozzle 12 having a bore 13 extending therethrough and communicating at one end, with the exterior of the reservoir and, at the other end, with the external environment. The exhaust end 14 of the nozzle 12 is placed in use by the operator so that the nozzle 12 is at a small acute angle relative to the surface of the scalp 15 somewhat as shown in FIG. 2 of the drawings. In a specific embodiment, to ensure that the solution is directed substantially only onto the scalp 15, the nozzle 12 has a relatively narrow outer diameter that can effectively penetrate between the hairs 16 growing from the scalp 15 as shown in FIG. 2.

In a specific embodiment, the nozzle may have a length of about 5.0 in. In another specific embodiment, the diameter of the nozzle bore may be about 0.030 in. In another specific embodiment, the outer diameter of the nozzle may be about 0.125 in. In another specific embodiment, the dimensions of the nozzle may include a nozzle length of about 5.0 in, a nozzle bore diameter of about 0.030 and an outer diameter of the nozzle of about 0.125 in.

When employing the heretofore-described atomizer 10 for treatment of human skin disorders, the rate of delivery of atomized solution to the scalp 15 may be between about 0.15 gm/sec. and about 0.8 gm/sec. Above about 0.8 g/sec., the rate of delivery of solution may be too rapid to provide substantially even coverage of the affected areas and may deliver an excess of solution onto the affected area. Below about 0.15 gm/sec., the rate of delivery may be too low to provide effective atomization of the solution. That is, below this minimum flow rate, the solution may be exhausted from the atomizer in drop or bulk form thereby “flooding” the treated skin area. In one specific embodiment of the present invention, the flow rate of solution may be between about 0.19 gm/sec. and about 0.4 gm/sec.

In another specific embodiment, nozzle bores of between 0.01 inches and about 0.06 inches also may be used depending upon the particular circumstances. The propellant pressure may be varied over a considerable range, depending upon the type of propellant used, which may include Non-CFC fluorocarbons such as heptafluoropropane, tetrafluoroethane, or the like. The net result is that the rate of delivery of atomized solution, to effect a satisfactory spray pattern, can vary.

The solution employed in the atomizer 10 is any effective solution which can be atomized using the delivery system of the invention. A solution for use in the previously-described atomizer 10 has the following composition:

| Component | % By Weight |
|---|---|
| Dexamethasone | 0.01 |
| Isopropyl myristate | 31.99 |
| Ethanol to make | 100% |

Another possible composition of may be the following:

| Component | % By Weight |
|---|---|
| Dexamethasone | 0.5 |
| Isopropyl myristate | 5.0 |
| Ethanol to make | 100% |

The solution may be atomized using an inert, nontoxic gas such as non-CFC fluorocarbons, for example, heptafluoropropane. It has been found advantageous to employ approximately 70 percent by weight of solution and 30 percent by weight gaseous propellant. For example, 70 grams of the above solution when combined with 30 grams of the propellant heptafluoropropane may be used.

In a specific embodiment, the compositions and methods of the present invention may include a minor amount of isopropyl myristate and a major amount of ethanol. As defined herein, “minor” amount means a lesser amount than the major amount and a “major” amount means an amount greater than the minor amount. In another specific embodiment, isopropyl myristate may be a minor amount and is about 31.99 percent by weight of the composition, less the weight of heptafluoropropane. In another embodiment, the compositions and methods of the present invention may include an effective amount of dexamethasone, and is about 0.01 percent by weight of the composition less the weight of heptafluoropropane.

In operation, the atomizer 10 is positioned near the scalp 15 so that the exit end of the nozzle 12 is almost touching the scalp, and so that the nozzle makes an acute angle with the scalp as shown in FIG. 2. Preferably, with the nozzle 12 angled as described, the nozzle is initially placed near the back of the scalp 15. The reservoir 11 is then activated, thereby forcing atomized, solution 17 from the nozzle 12 and onto the scalp 15. The nozzle 12 is then moved at a substantially uniform rate from its initial rear position to a position at the front of the scalp 15 along a line therebetween. While being so moved, the angle of the nozzle 12 relative to the scalp 15 is maintained substantially as above described. Maintaining the nozzle 12 in this position relative to the scalp 11 ensures uniform deposition of the solution and its active components on the scalp adjacent the nozzle's line of travel. When the nozzle 12 has reached the front of the scalp 15, the flow of solution from the atomizer 10 is discontinued.

To coat other areas of the scalp 15, the above procedure is preferably repeated. The atomizer 10 is moved over the scalp 15 in this manner until all of the affected areas of the scalp are covered with a substantially uniform coating of solution. As the atomizer 10 is moved over the scalp 15, the exit end 14 of the nozzle 12 moves correspondingly along a plane adjacent the scalp so that only the intended area receives solution and when hair is present, substantially little of the hair 16 is wetted by the solution.

The particular atomizer employed need not be of the push-button type, but may be, for example, of the squeezable type. Moreover, the squeezable type atomizer may he used for the delivering solution in atomized form at controllable rates such as those previously set forth. Additionally, the squeezable type atomizer may be used with a nozzle, or elongated nozzle as described.

Solutions employed for treatment of animal skin problems include anti-flea solutions containing pyrethrins and other chemicals in solution. Such solutions are atomized by the delivery system of this invention in the same manner as has just been described with respect to the treatment of human skin disorders.

This invention has been described with respect to the application to skin of a preparation for treating of certain skin conditions and ailments. However, included within the compass of this invention is the application of other preparations to the skin of animals, such as the anti-flea preparations.

What is claimed is:

1. A composition comprising a pressurized aerosol spray container having an actuator and containing a composition comprising an effective amount of an anti-inflammatory agent, and a carrier comprising non-volatile means for solubilizing said anti-inflammatory agent, a volatile organic compound and heptafluoropropane, and having a nozzle having a bore extending therethrough and having an exhaust end and an opposite end communicating with said container.

2. The composition of claim 1, wherein said volatile organic compound is ethanol.

3. The composition of claim 1 wherein said anti-inflammatory agent is a steroid.

4. The composition of claim 3, wherein said steroid is dexamethasone.

5. The composition of claim 1, wherein said solubilizing means is an ester derived from a lower alkyl alcohol and a fatty acid.

6. The composition of claim 5, wherein said alcohol is isopropyl alcohol.

7. The composition of claim 5, wherein said fatty acid is myristic acid.

8. A composition comprising a pressurized aerosol spray container having an actuator and containing a composition comprising, isopropyl myristate, ethanol, heptafluoropropane and an effective amount of dexamethasone, and having a nozzle having a bore extending therethrough and having an exhaust end and an opposite end communicating with said container.

9. The composition of claim 8, wherein an effective amount of dexamethasone is about 0.01 percent by weight of the composition less the weight of said heptafluoropropane.

10. The composition of claim 8, wherein 0.02 mg of dexamethasone is dispensed per second.

11. The composition of claim 8, wherein the amount of isopropyl myristate is about 31.99 percent by weight of the composition less the weight of said heptafluoropropane.

12. The composition of claim 8, wherein said nozzle has a length of about 5 inches and an outer diameter of about ⅛ inch.

13. A method of delivery of medicaments to human or animal skin areas comprising;
a pressurized aerosol spray container having an actuator and containing a composition comprising an effective amount of an anti-inflammatory agent, and a carrier comprising non-volatile means for solubilizing said anti-inflammatory agent, a volatile organic compound and heptafluoropropane, and having a nozzle having a bore extending therethrough and having an exhaust end and an opposite end communicating with said container; and
placing said exhaust end of said nozzle adjacent said skin areas; and
actuating said actuator such that the composition in said container is carried by said nozzle to said skin areas in atomized form, whereby said anti-inflammatory agent is concentrated in said non-volatile solubilizing means thereby facilitating absorption of the anti-inflammatory agent by said skin areas.

14. The method of claim 13, wherein said volatile organic compound is ethanol.

15. The method of claim 13, wherein said anti-inflammatory agent is a steroid.

16. The method of claim 15, wherein said steroid is dexamethasone.

17. The method of claim 13, wherein said solubilizing means is an ester derived from a lower alkyl alcohol and a fatty acid.

18. The method of claim 17, wherein said alcohol is isopropyl alcohol.

19. The method of claim 17, wherein said fatty acid is myristic acid.

20. A method of delivery of medicaments to human or animal skin areas comprising: a composition comprising a pressurized aerosol spray container having an actuator and containing a composition comprising isopropyl myristate, ethanol, heptafluoropropane and an effective amount of dexamethasone, and having a nozzle having a bore extending therethrough and having an exhaust end and an opposite end communicating with said container, and
placing said exhaust end of said nozzle adjacent said skin area; and
actuating said actuator such that the composition in said container is carried by said nozzle to said skin areas in atomized form, whereby said dexamethasone is concentrated in said isopropyl myristate thereby facilitating absorption of dexamethasone by said skin areas.

21. The method of claim 20, wherein an effective amount of dexamethasone is about 0.01 percent by weight of the composition less the weight of said heptafluoropropane.

22. The method of claim 20, wherein 0.02 mg of dexamethasone is dispensed per second.

23. The method of claim 20, wherein the amount of isopropyl myristate is about 31.99 percent by weight of the composition less the weight of said heptafluoropropane.

24. The method of claim 20, wherein said nozzle has a length of about 5 inches and an outer diameter of about ⅛ inch.
